(19) United States
(12) Patent Application Publication
(10) Pub. No.: US 2019/0209639 A1
(43) Pub. Date: Jul. 11, 2019

(54) Ointment for burn

(71) Applicants: Jeries Sami Ayoub, Riverside, CA (US); Mohammed Khair Abu Jadalla, El-Houson (JO)

(72) Inventors: Jeries Sami Ayoub, Riverside, CA (US); Mohammed Khair Abu Jadalla, El-Houson (JO)

(21) Appl. No.: 15/862,637

(22) Filed: Jan. 5, 2018

(51) Int. Cl. A61K 36/63; A61K 35/644; A61K 36/53

(52) U.S. Cl. CPC A61K 36/63; A61K 36/53; A61K 35/644

(57) ABSTRACT

Ointment for burn is made up of a special blend of natural ingredients, that is designed to aid in the healing process of first, second and third-degree burns.

This remarkable product has shown tremendous results when used as directed. Noticeable results appear in just a few days of applying the burn ointment to affected area.

Description;

Manner of making of the same;

These specifications details information to enable any person skilled in the art of science to which the invention or discovery appertains, or which it is most nearly connected, or make and use the same. As portion of the disclosure of the patent contains material which is subject to (copyright or mask work) protection. The copyright or mask work owner has no objection to the facsimile reproduction by anyone of the patent documents or the patent disclosure, as it appears in the patent and trademark office file or records, but otherwise reserves all (copyright or mask work) rights whatever. Submitted in compliance with 37 CFR 1.72 (b).

A61K35/00 Medicinal preparations containing materials or reaction products thereof with undetermined constitution from inanimate materials

This invention pertains to a medicinal preparation for the treatment of burns and other traumatic wounds.

Over the years, various methods have been worked out for the treatment of burns. All of these have had serious limitations.

The application of butesin picrate gave relief from pain, but was so toxic that often relatively minor burns terminated with death because of the absorption of the picrate toxins. Boric acid wet packs were less toxic, but did not prevent scarring and contractures. Tannic acid and silver nitrate were relatively ineffective.

A current method of burn treatment includes the steps of anaesthetizing the patient and scrubbing the burned area to cleanse it and remove debris. Thereafter skin grafts are applied. The latter treatment is disadvantageous since the mortality from shock is very high. Difficult and often unsuccessful skin grafting procedures axe necessary. Highly skilled medical personnel provided with elaborate equipment are required.

The net result is that at the present time the treatment of hums is non-uniform from place to place and the treatments applied are of minimum benefit. It has been said, in fact, that there has been made no real progress in the treatment of burns in the past several years.

### Purpose

First, second, and third degree skin burns are extremely dangerous and can take a considerable amount of time to heal. Treatments may include applying prescription creams and ointments to the skin. Some treatments may take an extended period of time, leaving people feeling uncomfortable. Some burns and rashes may require cosmetic surgery in order to heal the skin. Therefore, a composition made from natural ingredients used to heal skin burns is needed.

It is an important purpose of the present invention to provide a medicinal preparation for the treatment of burns which heals the burn section of the body quickly and efficiently, even in the case of third degree burns involving large skin areas; which is accompanied by a lower mortality rate; which brings great relief from the pain usually accompanying burns; and which results in healing of the burned area until it is covered with good skin with a minimum of scarring and contractures, and will not form any keloids on the skin. To effectively treat burned part(s) of the body, apply the ointment twice a day every 12 hours for certain time depending on the degree of the burn.

It is a further purpose of the present invention to provide a medicinal preparation generally useful in the promotion of healing of, and the prevention of infection in, traumatic wounds of classes other than burns, such as ulcerated wounds, amputations and other surgical wounds, abrasions, and the like.

The medicinal preparation, the use of which accomplishes the foregoing and other objects consists essentially of substantially a proportions by weight of olive oil, bees wax and marrubium vulgare (white horehound) leaves. The composition of matter to be employed depend upon such factors as the identity of the patient, the type of wound, the source and composition of the constituents, the identity of any fortifying agents which may be present, etc. However, in general, the three constituents may be used in the following proportions:

By weight olive oil 1000 grams, bees wax 200 grams, marrubium vulgare (white horehound) leaves 100 grams. A preferred composition for use in the treatment of burns comprises in percent about 76% olive oil and about 15% bees wax, and about 8% marrubium vulgare (white horehound) leaves by weight.

These three essential ingredients are used in amounts relative to each other sufficient to provide by weight of essential ingredients.

In the above formulation, the olive oil may be the readily available commercial USP grade of commercial olive oil. It serves dramatically the function of inhibiting granulation and the growth of proud flesh. The bees wax is used as a glazing agent or as a light/heat source, the marrubium vulgare (white horehound) leaves contains potent antimicrobial and anticancer properties.

The ointment can be used to treat acute burns, rashes, and the like.

The relative proportions of essential ingredients set forth in the formulation of the invention are critical to its successful use. Although in accordance with our discovery the marrubium vulgare (white horehound) leaves one of the primary active compounds found in horehound, to possess “antidiabetic, anti-atherogenic and anti-inflammatory properties”. The plant is reported to possess hypoglycemic, vasorelaxant, antihypertensive, analgesic, has the remarkable property of inhibiting granulation.

The wound to which the medicament has been applied initially may be covered with gauze and sheet wadding and thereafter dressed daily. At the time of dressing, all devitalized tissue and crusts which can be removed readily are removed. Tissue which is attached firmly is permitted to separate normally.

The patient may be permitted to walk about after the first days. He must not be permitted to keep any part of the body in abnormal flexion. To do so will produce contractures, as occurs with any bedfast who assumes an abnormal position.

At the end of the period of treatment, which may last for a period of several days to several weeks, the burned area will be covered with new skin which at first is thin and fragile, but which in time becomes of near normal thickness, healthy in appearance and soft and pliable. Disfiguring granulation tissue and contractures are substantially absent and the skin resumes its normal appearance.

The use of the medicinal preparation of our invention in the treatment of burns and other traumatic wounds thus is attended by manifold and significant advantages.

It promotes the healing of the burn by natural processes in dramatic fashion. It prevents infection, its use is attended by lower mortality rate, pain is diminished markedly, the formation of harmful granulation tissue is prevented, the wound area becomes covered over with good quality of pliable skin and infections developing on the wound area are minimized.

All of these advantages are obtained, furthermore, by the use of a preparation which is inexpensive, readily available and which may be applied by persons of ordinary medical training without the necessity of using elaborate and costly special equipment.

### DEFINITIONS, ACRONYMS, ABBREVIATIONS

Marrubium vulgare—(white horehound or common horehound) is a flowering plant in the mint family.
Olive oil—is a liquid fat obtained from olives, the oil is produced by pressing whole olives. It is commonly used in cooking.
Bees wax—is a natural wax produced by honey bees of the genus Apis.
Ointment—a smooth oily preparation that is rubbed on the skin for medicinal purposes or as a cosmetic.
Embodiment—a tangible or visible form of an idea.
Hypoglycemic—also known as low blood sugar.
Antimicrobial—is an agent that kills microorganisms or stops their growth.
Anticancer—An anticarcinogen (also known as a carcinopreventive agent) is a substance that counteracts the effects of a carcinogen or inhibits the development of cancer.
Antidiabetic—treat diabetes mellitus by lowering glucose levels in the blood.
Antiatherogenic—works against hardening of an artery specifically due to an atheromatous plaque.
Anti-inflammatory—refers to the property of a substance or treatment that reduces inflammation or swelling.
Hypoglycemic—also known as low blood sugar, is when blood sugar decreases to below normal levels.
Vasorelaxant—Vasodilation refers to the widening of blood vessels.^{[1]} It results from relaxation of smooth muscle cells within the vessel walls, in particular in the large veins (called venodilators), large arteries, and smaller arterioles.
Antihypertensive—is a class of drugs that are used to treat hypertension (high blood pressure). Antihypertensive therapy seeks to prevent the complications of high blood pressure, such as stroke and myocardial infarction.
Analgesic—or painkiller is any member of the group of drugs used to achieve analgesia, relief from pain.

### BRIEF DESCRIPTION OF DRAWINGS

FIG. 1 shows a perspective view of an embodiment of the burn treatment ointment stored in a jar, with a label has the writing of the ingredient on it.

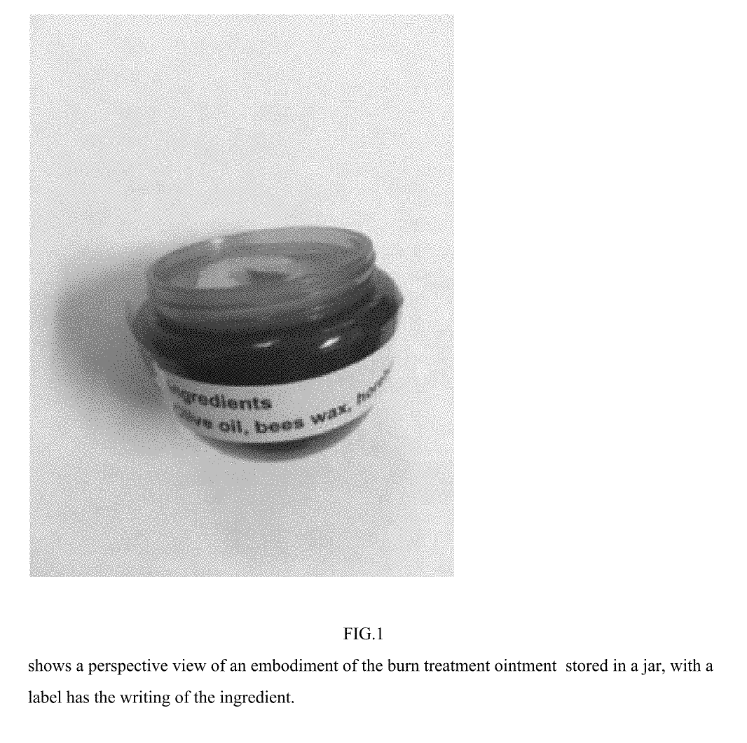

FIG. 2 shows a demonstrative view of the burn treatment ointment applied to a burn on a face.

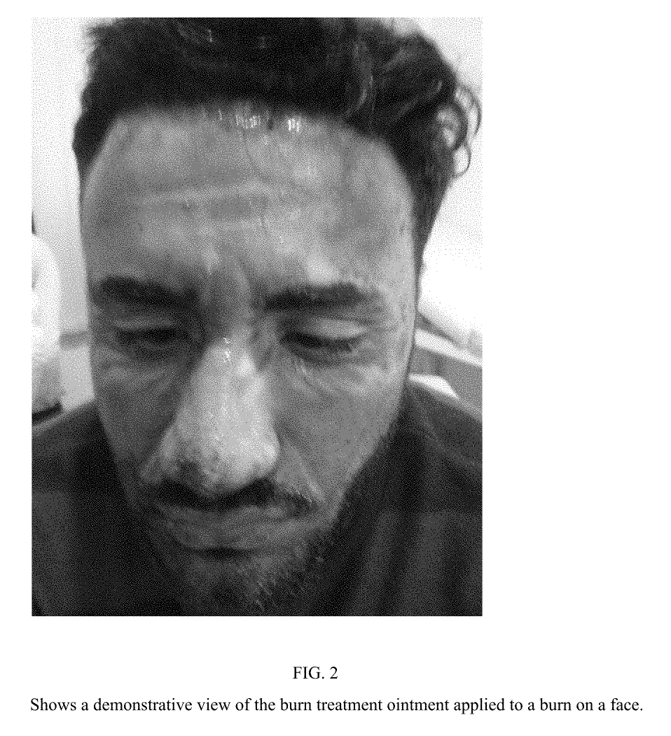

FIG. 3 shows a demonstrative view of the burn treatment ointment applied to a burn on an arm.

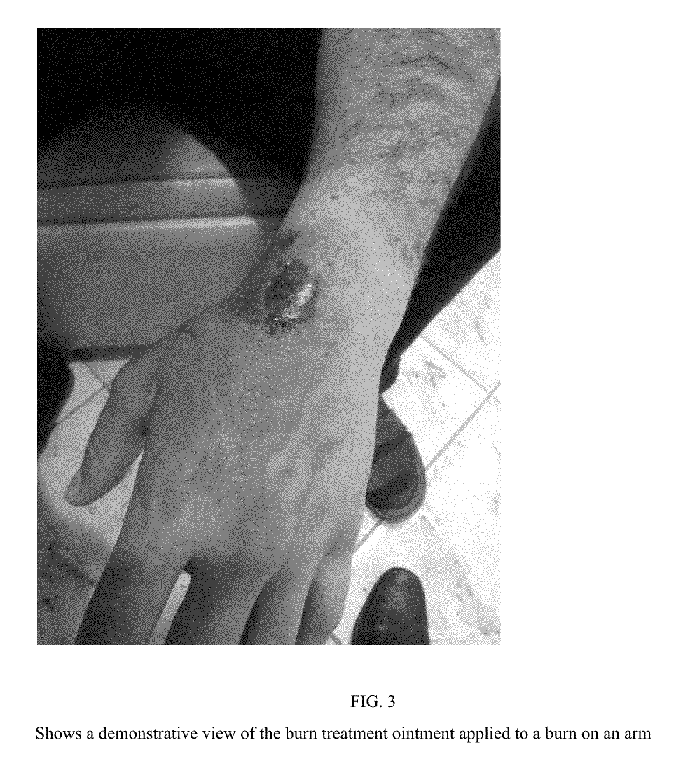

FIG. 4 shows a demonstrative view of a healed arm, the same arm in FIG. 3.

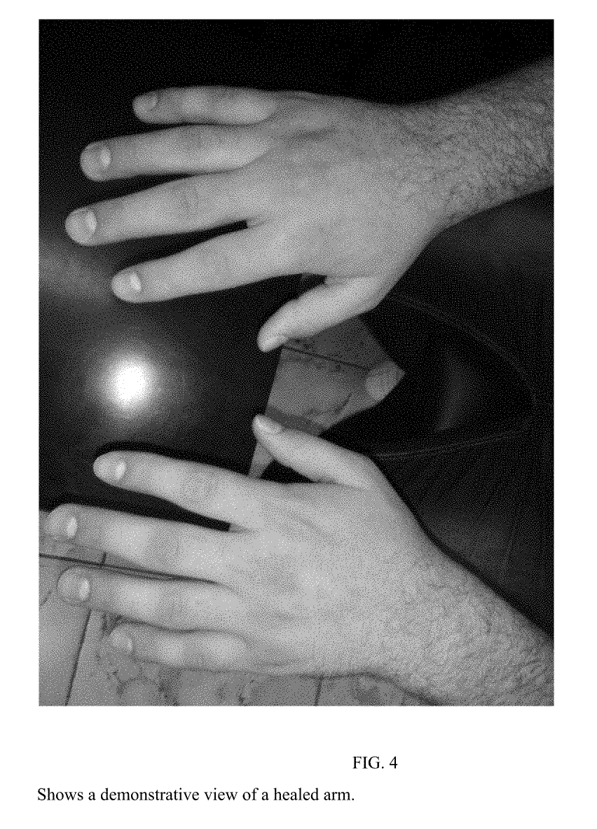

FIG. 5 show a demonstrative view of a healed face, the same face in FIG. 2

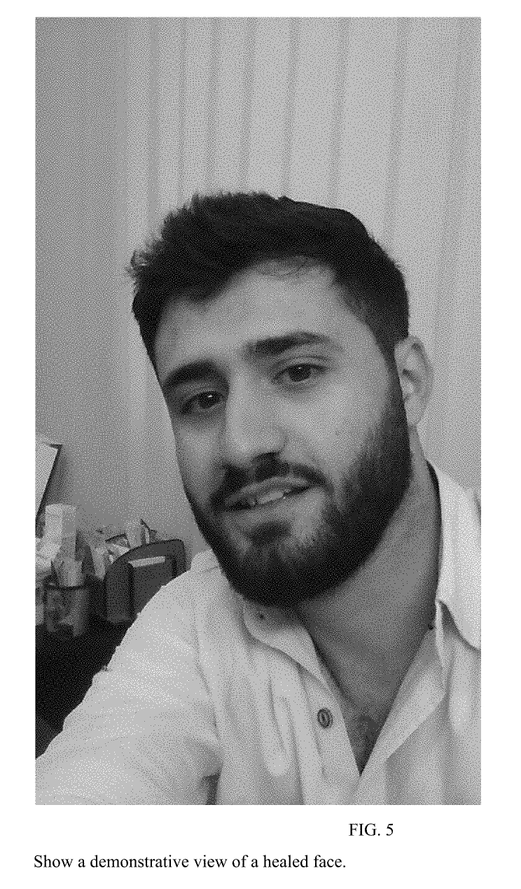

FIG. 6 shows a demonstrative view of the burn treatment ointment applied to a burn on a face.

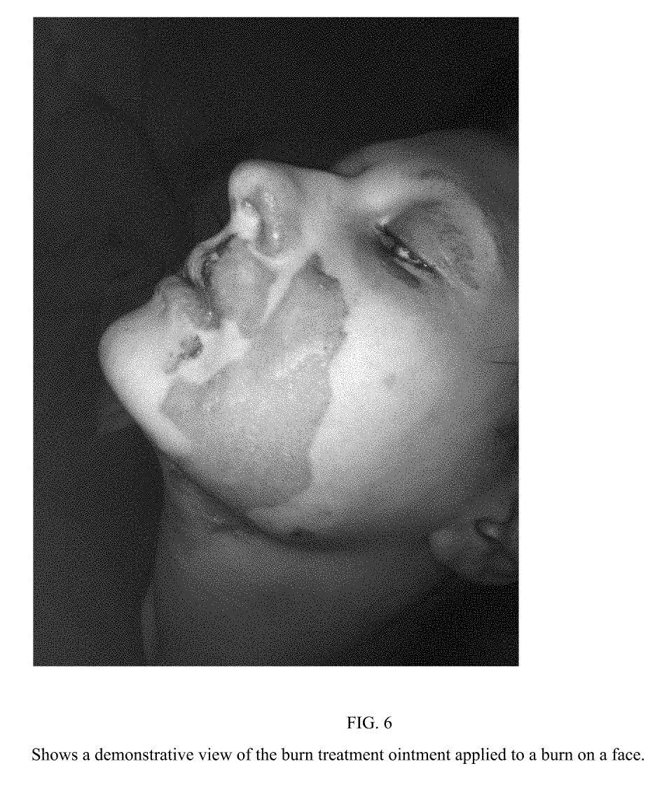

FIG. 7 show a demonstrative view of a healed face, the same face in FIG. 6

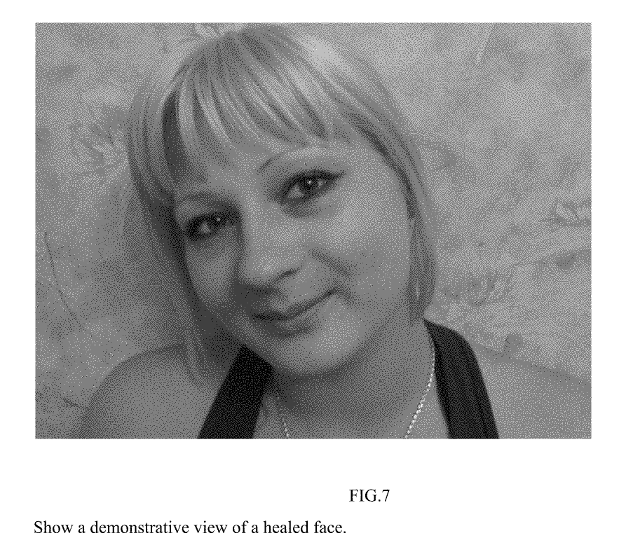

Having thus described our invention in preferred embodiments, we claim the following:

1. A medicinal preparation for the treatment of burns and other traumatic wounds, consisting essentially of:
The medicinal preparation of claim 1 wherein the essential constituents consist of about by weight olive oil 1000 grams, bees wax 200 grams, marrubium vulgare (white horehound) leaves 100 grams,
The medicinal preparation of claim 1 wherein the essential constituents consist of a proportion by percent olive oil to bees wax to marrubium vulgare (white horehound) leaves about 76-15-8 in order,
The medicinal preparation of claim 1 wherein the essential constituents consist of about percent by weight about 76% live oil, about 15% bees wax and about 8%. marrubium vulgare (white horehound) leaves.
The medicinal preparation of claim 1 wherein the essential constituents consist of about by weight olive oil 35.27 Oz, bees wax 7.05 Oz, marrubium vulgare (white horehound) leaves 3.53 Oz.
